(19) United States
(12) Patent Application Publication
(10) Pub. No.: US 2011/0305746 A1
(43) Pub. Date: Dec. 15, 2011

(54) PATCH FOR TREATING CONSTIPATION USING CRUDE DRUGS

(76) Inventor: Tae Jin Kim, Jeollabuk-do (KR)

(21) Appl. No.: 13/202,977

(22) PCT Filed: Feb. 19, 2010
(86) PCT No.: PCT/KR2010/001026

§ 371 Date: Aug. 23, 2011

(30) Foreign Application Priority Data
Feb. 23, 2009 (KR) 10-2009-0014979

(51) Int. Cl. A61K 9/70; B32B 37/14; B32B 37/12; A61K 36/708; A61P 1/10

(52) U.S. Cl. 424/449; 424/725; 156/326

(57) ABSTRACT

The present invention relates to a patch for treating constipation, which contains rhubarb ingredients selected from among crude drugs (medical herbs). More particularly, the present invention relates to a patch for treating constipation in which powder obtained by boiling, filtering, and free-drying rhubarb selected from among pure crude drugs is applied to the patch, and the patch is attached to the recesses (arched parts) below the wrists of both hands of a patient, to thereby increase the metabolism of internal organs of the patient and exhibit the effects of treating or preventing constipation. The patch of the present invention can be simply attached to the skin of the patient instead of directly administering agents for treating constipation to the patient.

### TECHNICAL FIELD

The present invention relates to a patch for treating constipation. More particularly, the present invention relates to a patch for treating constipation using crude drugs (medical herbs) having constipation treatment effect wherein powder obtained by boiling, filtering and freeze-drying one or more rhubarb ingredients selected from the group consisting of Rheum palmatum L., RHEUMTANQUTICUM MAXIM ET Baif, Rheum undulatum L., Rheum Officinale Baill., Rheum coreanum Nakai, and Rhei Undulatai Rhizoma is applied to a patch and wherein the patch is attached to the recesses (arched parts) below the wrists of both hands of a patient, to thereby activate the function of internal organs of the patient through acupoint and increase its metabolism.

### 2. Background Art

As an industry is advanced, a population aging is rapidly proceeded and an automobile culture is gradually spread, a lack phenomenon of physical activity of the body is aggravated. Therefore, the number of patients with constipation is greatly increased.

Constipation means that passage of waste at a lower part of large intestine is delayed or hard and that dry stools are discharged through the anus. When a patient is constipated, the patient has conditions such as a feeling of fullness, a rectal pressure and a desire for frequent excretion.

Particularly, in case of a chronic constipation, patients with chronic constipation have epigastric sense of distention, eructation, kinesia, reflux, and abdomen dysphoria. If chronic constipation is not treated, patients with chronic constipation may have a general symptom such as headache, dizziness, loss of appetite, stomatodysodia, and skin roughness.

However, most patients with constipation feel shyness or they think that constipation treatment itself is very complicated.

Furthermore, most patients with constipation tend not to quickly treat constipation. The reason is that the patients with constipation do not think that most constipation is serious diseases.

As the method for treating constipation, it is preferable to recover and maintain, as usual, regular meals and regular bowel habits, or to increase the uptake of waters and foods having a lot of moisture content and fiber, e.g., fresh fruits and vegetables, not refined grains and the like.

Also, it is effective to get a regular physical activity rather than to take agents for treating constipation. For persons with weak muscle tension, it is effective to get an abdominal exercise.

The inventor of the present application has conducted extensive searches and researches about how to effectively treat constipation using crude drugs (medical herbs). As a result, the inventor has discovered that rhubarb among the crude drugs is very effective in treating constipation.

Rhubarb, one of Polygonum aviculare L. plants, contains anthraquinone derivatives and tannin ingredients in its root and rhizome. Therefore, rhubarb is a crude drug (medical herbs) which assists a peristaltic movement of intestine and which is used for treating constipation. Root extract of rhubarb has been known to have a suppression activity against E. coli, but its efficacy is not used in the art (see, Ingredients and Utilization of Medical Herbs, Ilworlseogak, 1994, Scientific Encyclopedia Publisher, Choongyak Unabridged Dictionary, Jeongdam Publishing Company, Choongyak Unabridged Dictionary Compilation Committee, 1997).

As prior art references using rhubarb, Korean Unexamined Patent Publication No. 10-2001-18655 discloses a cosmetic composition; Korean Patent Publication No. 180994 discloses a soap composition; Korean Patent No. 361040 discloses a cosmetic composition; and Korean Unexamined Patent Publication No. 10-2003-55154 discloses a composition for treating a skin disease. However, these references merely mention that rhubarb is used in a cosmetic composition or a composition for treating skin diseases, there being no disclosure concerning the use of rhubarb for treatment of constipation.

Rhubarb is classified into more than six kinds including Rheum palmatum L., RHEUMTANQUTICUM MAXIM ET Baif, Rheum undulatum L., Rheum Officinale Baill., Rheum coreanum Nakai, and Rhei Undulatai Rhizoma.

Regarding the efficacies of rhubarb, the property is very cold, the taste is bitter and it has no toxicity. Rhubarb is effective in treating extravasated blood and menstrual irregularity. Further, rhubarb is effective in treating congestion constipation and fever constipation. For example, Rhubarb is effective in well passing faeces and urine, and cooling heat.

In Chinese medicine, rhubarb has been used as an inflammatory purgative from the pre-Christ era and in combination with other ingredients in several prescriptions. The main ingredients of rhubarb include anthraquinone derivatives and glycoside thereof, and tannin. Taking rhubarb in a small amount exhibits a stomachic action, and taking it in a large amount is useful in treating habitual constipation or dyspepsia. It has also been known to be used for a burn treatment as folk drugs.

However, there were problems in that general medical herbs must be decocted and eaten into a soup. That is, although rhubarb among medical herbs has an excellent efficacy in treating constipation, it must be mixed and prepared with other medical herbs, and decocted and eaten in a proper amount. Therefore, there has been an inconvenience that, whenever taking the drugs, the process for decocting them has to involve.

In discussing Western medicine and Chinese medicine (herb medicine), since Chinese medicine rather than Western medicine is a herb medicine, its treatment effect is confident and the side effect is low. Chinese medicine is more excellent in a fundamental treatment handling a physical constitutional improvement or internal organs than Western medicine. Nevertheless, Chinese medicine in the form of a medicated patch or a patch attaching to the portions of muscle pain, arthralgia and neuralgia has not yet been developed. Therefore, there was a problem that it is not possible to treat the pains in the form of attaching Chinese medicine (herb medicine) to a skin.

Particularly, there was a problem that since constipation treatment has been made in an oral administration route until now, excessive drugs have been used. The present invention has been developed a patch comprising rhubarb ingredient which can treat constipation by locally attaching a patch to a skin, without taking Chinese medicine.

### DISCLOSURE OF INVENTION

### Technical Solution

The purpose of the present invention is to resolve the above mentioned problems and to provide a patch for treating constipation using crude drugs (medical herbs) having constipation treatment effect. The patch comprising rhubarb ingredient according to the present invention can treat constipation simply and easily by attaching the patch to the recesses (arched parts) below the wrists of both hands of a patient, without taking rhubarb ingredient among naïve crude drugs.

### Technical Solution

In order to accomplish the above purpose, the present invention provides a patch comprising rhubarb ingredient prepared by comprising the following steps:

clearly washing and drying rhubarb, boiling it at a temperature of 90˜110° C. for 3˜10 hours to separate a liquid ingredient;

keeping and precipitating the above separated liquid rhubarb extract ingredient in a storeroom at a temperature of 3˜5° C. for 5˜10 days;

completely filtering the resulting ingredient with 120˜200 mesh filter paper and centrifuge to give a liquid Rhubarb concentrate and then freeze-drying the concentrate to prepare a rhubarb extract powder, and then adding a solvent, an adhesive and the like to the rhubarb extract powder to prepare a solution;

coating a rubber based adhesive on a patch cloth at a thickness of 0.02˜0.04 mm and then drying it;

coating the above prepared solution on a release paper at a thickness of 0.05˜0.1 mm and then drying it; and

bonding the cloth and the release paper and then performing lamination so that the coating layers can correspond to each other.

Specifically, the process for preparing the patch for treating constipation according to the present invention comprises the following steps:

mixing the freeze-dried rhubarb extract powder and water in a ratio of 1:1.2 and then dissolving the mixture in N-methyl-2-pyrrolidone (NMP);

adding Povidone K90, Povidone K25 and ethanol to the above solution and sufficiently mixing them with stirring by a stirrer for 24 hours;

adding sorbitan monooleate (SMO), acrylic adhesive solution (BA2000) and butylated hydroxytoluene (MT) to the prepared solution and mixing them;

coating a rubber based adhesive on a patch cloth and then drying it;

coating the above prepared drug solution on a release paper at a regular thickness and then drying it;

bonding the cloth and the release paper, performing lamination at the temperature of 50° C. and cutting them in a desired size to prepare a patch; and

attaching the patch to the recesses (arched parts) below the wrists of both hands of a patient once a day (12˜24 hours) for more than 2 days.

As set forth above, according to the present invention, constipation can simply and easily be treated by locally attaching the patch for constipation treatment comprising rhubarb ingredient to a skin of a patient, without taking the rhubarb ingredient.

### Effect of Invention

According to the present invention, constipation can effectively be treated by preparing the patch comprising rhubarb and then attaching the patch to the recesses (arched parts) below the wrists of both hands of a patient for 12˜24 hours. Also, the patch has the effect of preventing, in advance, constipation in persons who are frequently constipated.

The patch comprising rhubarb ingredient and the method for treating constipation according to the present invention are effective in treating constipation by simply attaching the patch to a skin, without taking agents for treating constipation.

### BEST MODE FOR CARRYING OUT THE INVENTION

Hereinafter, the embodiments of the present invention will be described in detail.

The patch comprising rhubarb ingredient according to the present invention is prepared by comprising the following steps:

step of clearly washing and drying rhubarb, boiling it at a temperature of 90˜110° C. for 3˜10 hours to separate a liquid rhubarb ingredient;

step of keeping and precipitating the above separated liquid rhubarb extract ingredient in a storeroom at a temperature of 3˜5° C. for 5˜10 days;

step of completely filtering the above precipitated ingredient with 120˜200 mesh filter paper and centrifuge to give a rhubarb concentrate;

step of freeze-drying the above filtered rhubarb concentrate to prepare a rhubarb powder;

step of mixing 492 g of the freeze-dried rhubarb powder with 500˜600 cc of water and then dissolving it in 500˜600 cc of N-methyl-2-pyrrolidone (NMP) to prepare a solution;

step of adding 70˜90 g of Povidone K90, 70˜90 g of Povidone K25 and 500˜600 cc of ethanol to the above solution and sufficiently mixing them;

step of adding 235˜255 g of sorbitan monooleate (SMO), 3500˜4000 cc of acrylic adhesive solution and 10˜15 g of butylated hydroxytoluene (BHT) to the above mixed solution and mixing them to prepare a patch solution;

after preparing the patch solution as above, step of coating a rubber based adhesive on a patch cloth at a thickness of 0.02˜0.04 mm and then drying it;

step of coating the above prepared patch solution on a release paper at a thickness of 0.05˜0.1 mm and then drying it; and

step of bonding the cloth coated with the rubber adhesive and the release paper coated with the patch solution, and then performing lamination.

Also, the present invention provides the process for preparing the patch for treating constipation comprising one or more rhubarb ingredients selected from the group consisting of Rheum palmatum L., RHEUMTANQUTICUM MAXIM ET BAIF, Rheum undulatum L., Rheum Officinale Baill., Rheum coreanum Nakai., and Rhei Undulatai Rhizoma.

According to the other embodiment of the present invention, the patch is prepared by comprising the following steps:

step of mixing the boiled, filtered and freeze-dried rhubarb extract powder and water in a ratio of 1:1.2 and then dissolving the mixture in N-methyl-2-pyrrolidone (NMP);

step of adding Povidone K90, Povidone K25 and ethanol to the above dissolved solution and stirring them so as to be sufficiently mixed for 24 hours;

step of adding and mixing sorbitan monooleate (SMO), acrylic adhesive solution (BA2000) and butylated hydroxytoluene (BHT) to the solution prepared by mixing for 24 hours to prepare a patch solution;

after preparing the patch solution, step of coating a rubber based adhesive on a patch cloth and then drying it;

step of coating the above prepared patch solution on a release paper at a regular thickness and then drying it; and

step of bonding the cloth and the release paper, conducting lamination at the temperature of 50° C. and cutting them in a desired size to prepare a patch.

The patch for treating constipation comprising rhubarb ingredient among crude drugs is attached and used to the recesses (arched parts) below the wrists of both hands of a patient once a day (12˜24 hours) for more than 2 days. However, the patch is characterized by treating constipation in the manner of skipping one day, attaching one day, skipping two days, attaching one day and skipping three days, from the next day that excretion is smoothly made.

Constipation to be treated in the present invention means that stools having no water are laboriously eliminated from the body. In contrast, eliminating stools having a lot of moisture from the body is diarrhea. That is, constipation has no water in bowel movement.

In case of persons having a lot of heat and angry, the moisture will disappear. However, in case of persons having a lot of cooling, the more moisture will be formed.

Senile constipation results from lack of juice, but the juice is also made of moisture. Therefore, cooling heat and supplementing moisture is a basis for constipation treatment.

For the patch for treating constipation comprising rhubarb ingredient among crude drugs to treat constipation in the present invention, the rhubarb ingredient does not simply cool only heat, but it increases moisture and supplements juice.

Rhubarb used herein is selected from the group consisting of Rheum palmatum L., RHEUMTANQUTICUM MAXIM ET BAIF, Rheum undulatum L., Rheum Officinale Baill., Rheum coreanum Nakai., and Rhei Undulatai Rhizoma. Among the above rhubarb ingredients, Rheum palmatum L. or Rhei Undulatai Rhizoma exhibits an excellent effect in constipation treatment.

In order to prepare the patch for treating constipation comprising rhubarb according to the present invention, rhubarb is boiled, safely filtered with a filter paper and a centrifuge and then freeze-dried to prepare a rhubarb extract powder.

The examples of the present invention are described in detail step-by-step.

First step: Separation of liquid ingredient from Rhubarb

Rhubarb is clearly washed, dried and then boiled at a temperature of 90˜110° C. for 3˜10 hours to separate a liquid rhubarb ingredient.

The above selected and regularly dried rhubarb is boiled in a conventional bath at a temperature 90˜110° C. for 3˜10 hours. Depending on the amount of the rhubarb, it is boiled in a bath for between 3 hours and 10 hours to separate a liquid ingredient.

Second step: Precipitation

The liquid rhubarb ingredient separated as a liquid ingredient in the first step is kept and precipitated in a storeroom at a temperature of 3˜5° C. for 5˜10 days.

The rhubarb solution separated as the liquid ingredient is precipitated and concentrated in the course of being stored at a temperature of 3˜5° C. for 5˜10 days. A fine sludge is precipitated and a clear solution is placed at the upper portion. In case of storing for less than five days, the time of precipitating the fine sludge is insufficient. In case of precipitating for more than ten days, the precipitation is not made any longer and the denaturation is initiated. It is preferable that the liquid rhubarb is precipitated in a storeroom for 5˜10 days.

Third step: Freeze-drying

The liquid Rhubarb precipitated in a storeroom as above is completely filtered with 120˜200 mesh filter and centrifuge to give a liquid rhubarb concentrate. The resulting concentrate is then freeze-dried to prepare a rhubarb extract powder.

The freeze-drying is carried out according to a conventional freeze-drying method. The rhubarb concentrate is freeze-dried in this manner and powdered.

Fourth step: Primary Dissolution

In order to prepare the resulting rhubarb extract powder in the form of a patch, solvent, adhesive and the like are further added.

To 492 g (based on part by weight) of the freeze-dried rhubarb extract powder, 500˜600 cc of water is added and mixed. The mixture is then dissolved in 500˜600 cc of N-methyl-2-pyrrolidone (NMP).

To the above dissolved solution, 70˜90 g of Povidone K90 (polymer material for increasing a compatibility of crude drugs and adhesive), 70˜90 g of Povidone K25 (polymer material for increasing a compatibility of crude drugs and adhesive) and 500˜600 cc of ethanol are added.

In order to sufficiently mix the above mixture, the mixture is stirred and mixed for 24 hours.

Fifth step: Secondary Dissolution

To the solution prepared as above, 235˜255 g of sorbitan monooleate (SMO: material for increasing skin permeability of drugs), 3500˜4000 cc of acrylic adhesive solution (BA2000: product name) and 10˜15 g of butylated hydroxytoluene (EHT) are added and mixed. The mixture is stirred for a sufficient mixing for 3˜5 hours to prepare a patch solution.

Sixth step: Preparation of Patch

After the patch solution is prepared as above, a rubber based adhesive is coated on a patch cloth at a thickness of 0.02˜0.04 mm.

Also, the patch solution prepared thus is coated on a release paper at a thickness of 0.05˜0.1 mm and then dried.

The cloth coated with rubber based adhesive is bonded with the release paper coated with rhubarb solution (patch solution) and lamination is performed at a temperature of 50° C. so that the coated layers can correspond to each other.

The patch formed as above is cut and packed in accordance with a conventional standard to prepare a final patch product.

In the other embodiment of the present invention, the liquid rhubarb is completely filtered and then freeze-dried to give rhubarb extract powder. Rhubarb extract powder and water are mixed in a ratio of 1:1.2. The mixture is then dissolved in N-methyl-2-pyrrolidone (NMP).

To the above dissolved solution, Povidone K90, Povidone K25 and ethanol are mixed. First stirring of the above mixture is performed so as to be sufficiently mixed for 24 hours. In the subsequent step, to the solution dissolved in N-methyl-2-pyrrolidone (NMP), sorbitan monooleate (SMO), acrylic adhesive solution (BA2000) and butylated hydroxytoluene (BHT) are added in stirrer, and second stirring is performed so as to be sufficiently mixed for 3˜5 hours.

After the patch solution is prepared as above, a rubber based adhesive is coated on a patch cloth and dried. The patch solution prepared thus is coated on a release paper at a regular thickness and then dried.

The cloth coated with rubber based adhesive is then bonded with the release paper coated with rhubarb solution (patch solution) and lamination is performed at a temperature of 50° C. so that the coated layers can correspond to each other.

The patch cloth formed as above is cut in accordance with a conventional standard to prepare a patch.

The patch is cut and packed by a size which is conventionally available in the market.

The patch formed as above is attached and used to the recesses (arched parts) below the wrists of both hands of a patient with constipation once a day (12˜24 hours) for more than 2 days.

That is, the patch formed as above is attached and used to the recesses (arched parts) below the wrists of both hands once a day (12˜24 hours) for more than 2 days, but constipation is treated in the manner of skipping one day, attaching one day, skipping two days, attaching one day and skipping three days, from the next day that excretion is smoothly made.

The composition comprising the rhubarb solution used as the agents for treating constipation and the dissolving agent in the present invention are formulated as shown in Table 1 below:

TABLE 1

| Material name | Mixing Amount (g) |
|---|---|
| Rhubarb extract powder | 492.00 |
| Sorbitan monooleate (SMO) | 246.00 |
| Povidone K90 | 82.00 |
| Povidone K25 | 82.00 |
| Butylated hydroxytoluene (BHT) | 12.30 |
| Ethanol (EtOH) | 549.40 |
| N-methyl-2-pyrrolidone (NMP) | 549.40 |
| Water | 549.40 |
| Acrylic adhesive solution (BA2000) | 3751.50 |

Here, sorbitan monooleate (SMO) is a non-ionic surfactant for increasing skin permeability of drugs. N-methyl-2-pyrrolidone (NMP) is a solvent for excellently dissolving several water-insoluble drugs. Povidone K90 and Povidone K25 are polymer materials for increasing compatibility of crude drugs and adhesives. Butylated hydroxytoluene (BMT) is an anti-oxidizing agent.

492 g of Rhubarb extract powder is regularly dissolved in 549.40 cc of water and 549.40 cc of N-methyl-2-pyrrolidone (NMP).

To the solution in which rhubarb extract powder is dissolved, 82.00 g of Povidone K90 and 82.00 g of Povidone K25 (polymer material for increasing a compatibility of crude drugs and adhesive) and 549.40 cc of ethanol are added and mixed. The mixing time is about one day.

To the solution prepared as above, 246.00 g of sorbitan monooleate (SMO: non-ionic surfactant for increasing skin permeability of drugs), 3751.50 cc of acrylic adhesive solution (BA2000) and 12.30 g of butylated hydroxytoluene (BMT) are added and mixed.

Separately, a rubber based adhesive is coated on a patch cloth at a thickness 0.02˜0.04 mm and dried.

The patch solution prepared in the above is coated on a release paper at a thickness of 0.05˜0.1 mm and then dried.

The cloth coated with the rubber based adhesive is bonded with the release paper coated with the patch solution and lamination is performed at a temperature of 50° C. to prepare a patch cloth containing rhubarb ingredient.

The patch cloth is cut and packed by a desired size to prepare a final patch product.

### MODE FOR CARRYING OUT THE INVENTION

Examples which have clinically tested to patients with constipation using the patch for treating constipation according to the present invention prepared as above are reviewed and noted as follows:

### Clinical Test Example

Test was performed in the name of Chungsan Saengyak Institute (president: the applicant of the present application) at the Health Centers of Keunyoung Women's High School and Jeonbuk National University in Korea. The patch comprising rhubarb ingredient as described above was attached and used to the recesses (arched parts) below the wrists of both hands of 180 patients with constipation once a day for 12˜24 hours and used for 2 days. The test result was explained according to “Analysis of the Answer to Questionnaire from the User of Constipation Drugs for Attachment to Skin”.

The content of questionnaire on constipation drugs was as follows:

1. Regarding the question, how long are you constipated?

Answered: \textcircled{1} “less than one year” was about 30% (54 persons), \textcircled{2} “one year” was about 30% (56 persons), \textcircled{3} “two year” was about 30% (50 persons), and \textcircled{4} “more than three years” was about 10% (20 persons).

2. Regarding the question, how is your constipation condition?

Answered: \textcircled{1} “a little severe” was about 80% (140 persons), \textcircled{2} “too severe” was about 15% (30 persons), and \textcircled{3} “feel like crying” was about 5% (10 persons).

3. Regarding the question, did you attach the patch on your skin and go to stool?

Answered: \textcircled{1} “went to stool within 12 hours” was about 60.00% (110 persons), and \textcircled{2} “went to stool within two days” was about 40% (70 persons). Therefore, the patch of the present invention has exhibited the effect of going to stool within two days after the attachment thereof.

4. Regarding the question, how do you feel about going to stool?

Answered: \textcircled{1} “went to moderate stool” was about 70.00% (130 persons), \textcircled{2} “went to a lot of stool” was about 10% (20 persons). \textcircled{3} “went to stool to cool” was about 15% (24 persons), and \textcircled{4} “very satisfied” was about 5% (6 persons). The users were favorable to the use result of the patch of the present invention.

The patch according to the present invention has shown the best effect on the constipation treatment when the patch was applied to the recesses (arched parts) below the wrists of both hands of a patient.

The recesses (arched parts) below the wrists of hands are the place deeply set at the lowest portion that does not appear when the wrists are straight. In anatomy, they are the recesses between ulnas styloid process and triangular bone at the ulnar edge of the dorsal arm striation.

As the other treatment method of the present invention, the test result has shown that the patch of the present invention was effective even in the recesses of the upper portion of the wrists and the portions of abdominal left transverse colon and descending colon. However, such effect was not greater than when the patch was attached to the recesses of the lower portion of the wrists.

The patch of the present invention has exhibited excellent effect in constipation treatment by attaching the patch to a skin, without taking agents for treating constipation. However, patients with chronic constipation have to intermittently attach the patch of the present invention to the skin and consistently treat constipation.

### INDUSTRIAL APPLICABILITY

According to the present invention, constipation can effectively be treated by preparing the patch comprising rhubarb ingredient and attaching the patch to the recesses (arched parts) below the wrists of both hands of a patient for 12˜24 hours. Therefore, by adding the method for treating constipation from the patch in addition to a conventional constipation treatment method, patients can select treatment methods suitable for their physical characteristics in hospital and drugstore. Further, the patch has the effect of preventing, in advance, constipation in persons who are frequently constipated.

The patch comprising rhubarb ingredient and the method for treating constipation according to the present invention are very effective in treating constipation by simply attaching the patch to a skin, without taking agents for treating constipation. The patch of the present invention can be simply attached to the skin of the patient, without orally administering agents for treating constipation which may cause obstacles in stomach and intestine.

What is claimed is:

1. A patch for constipation treatment using crude drugs which comprises the following steps:
step of clearly washing one or more rhubarb ingredients selected from the group consisting of Rheum palmatum L., RHEUMTANQUTICUM MAXIM ET BAIF, Rheum undulatum L., Rheum Officinale Baill., Rheum coreanum Nakai., and Rhei Undulatai Rhizoma, uniformly drying and boiling it at a temperature of 90˜110° C. for 3˜10 hours to separate a liquid rhubarb extract ingredient;
step of keeping and precipitating the above separated liquid rhubarb extract ingredient in a storeroom at a temperature of 3˜5° C. for 5˜10 days;
step of completely filtering the above precipitated ingredient with 120˜200 mesh filter and centrifuge to give a liquid rhubarb concentrate;
step of freeze-drying the above filtered concentrate to prepare a rhubarb extract powder;
step of mixing 492 g of the above freeze-dried rhubarb powder with 500˜600 cc of water and then dissolving it in 500˜600 cc of N-methyl-2-pyrrolidone (NMP);
step of adding 70˜90 g of Povidone K90, 70˜90 g of Povidone K25 and 500˜600 cc of ethanol to the above resulting solution and stirring them so as to be sufficiently mixed;
step of adding 235˜255 g of sorbitan monooleate (SMO), 3500˜4000 cc of acrylic adhesive solution and 10˜15 g of butylated hydroxytoluene (BHT) to the above mixed solution and mixing them to prepare a final patch solution;
after preparing the patch solution as above, step of coating a rubber based adhesive on a patch cloth at a thickness of 0.02˜0.04 mm and then drying it;
step of coating the above prepared final patch solution on a release paper at a thickness of 0.05˜0.1 mm and then drying it; and
step of bonding the cloth coated with the rubber adhesive and the release paper coated with the patch solution, and then performing lamination to prepare a final patch.

2. The patch for constipation treatment using crude drugs according to claim 1 wherein the powder prepared by boiling, filtering and freeze-drying one or more rhubarb ingredients selected from the group consisting of Rheum palmatum L., RHEUMTANQUTICUM MAXIM ET Baif, Rheum undulatum L., Rheum Officinale Baill., Rheum coreanum Nakai. and Rhei Undulatai Rhizoma is applied to a patch and wherein the patch is attached to the recesses (arched parts) below the wrists of both hands of a patient, to thereby activate the function of internal organs of the patient and increase its metabolism.

3. The patch for treating constipation using crude drugs according to claim 1 which comprises the following steps:
step of mixing the boiled, filtered and freeze-dried rhubarb extract powder and water in a ratio of 1:1.2 and then dissolving the mixture in N-methyl-2-pyrrolidone (NMP);
step of adding Povidone K90, Povidone K25 and ethanol to the above dissolved solution and precipitating and stirring so as to be sufficiently mixed for 24 hours;
step of adding sorbitan monooleate (SMO), acrylic adhesive solution (BA2000) and butylated hydroxytoluene (BHT) to the above mixed solution and mixing them to prepare a patch solution;
after preparing the patch solution as above, step of coating a rubber based adhesive on a patch cloth and then drying it;
step of coating the above prepared patch solution on a release paper at a regular thickness and then drying it; and
step of bonding the cloth and the release paper, conducting lamination at the temperature of 50° C. and cutting them in a desired size to prepare a final patch.
